(19) United States
(12) Patent Application Publication
(10) Pub. No.: US 2025/0087341 A1
(43) Pub. Date: Mar. 13, 2025

(54) MULTIMODAL MANAGEMENT OF PATIENT INTERACTIONS

(71) Applicant: Optum, Inc., Minnetonka, MN (US)

(72) Inventors: Brandon Matthew Brown, Knoxville, TN (US); Srilekha Akula, Fremont, CA (US); Stephen Ford Chambers, Northlake, TX (US); Timothy Meindle, Mt. Prospect, IL (US); Chandrakanth Reddy Mamillapalli, Flagstaff, AZ (US); Andras Ferenczi, Scottsdale, AZ (US)

(21) Appl. No.: 18/466,140

(22) Filed: Sep. 13, 2023

(51) Int. Cl. G16H 40/20; G16H 40/67

(52) U.S. Cl. CPC G16H 40/20; G16H 40/67

(57) ABSTRACT

A system and methods for multimodal patient management in a healthcare facility are disclosed. The method can include determining that a patient is within a threshold distance of a healthcare facility based on geolocation data for a wearable device associated with the patient, automatically checking-in the patient for an appointment; transmitting, to the wearable device, a notification indicating: i) that the patient ready to be seen the healthcare provider for the scheduled appointment, and ii) a target area in the healthcare facility to which the patient is to navigate for the scheduled appointment; continuously tracking a position of the patient within the healthcare facility using the wearable device, in part to determine when the patient has exited the healthcare facility; and updating a status of the patient in a digital patient tracker based on the tracked position of the patient within the healthcare facility.

### BACKGROUND

Healthcare facilities, such as hospitals, clinics, and the like, are often subject to a continuous influx of patients seeking medical services and care. Traditionally, the patient check-in process at these facilities involves patients manually filling out paper forms with their personal and medical information, presenting identification documents, and waiting in long queues to register for appointments. This conventional check-in procedure is often time-consuming, cumbersome, and prone to errors, leading to delays in patient processing, increased administrative burdens, and diminished patient satisfaction.

To address some of these challenges, various check-in systems have been developed and implemented. For instance, some facilities have adopted electronic kiosks allowing patients to enter their information and check-in digitally. While such solutions offer some improvements over paper-based processes, they may still require patients to physically interact with kiosks—which are typically located within the healthcare facility—and could result in potential data privacy concerns if not adequately managed. Moreover, existing solutions typically do not provide feedback to patients after check-in, nor do they actively manage patients to avoid congestion and other issues. In one example, patients typically do not have a convenient way to inform the healthcare facility's staff of their status and location, which can cause delays for the healthcare facility and reduced patient satisfaction.

### SUMMARY

One implementation of the present disclosure is a patient management system including: one or more processors; and memory having instructions stored thereon that, when executed by the one or more processors, cause the system to: determine that a patient is within a threshold distance of a healthcare facility based on geolocation data for a wearable device associated with the patient, wherein the patient has a scheduled appointment with a healthcare provider at the healthcare facility; automatically check-in the patient for the scheduled appointment once it is determined that the patient is within the threshold distance of the healthcare facility; transmit, to the wearable device associated with the patient, a first notification indicating: i) that the patient ready to be seen the healthcare provider for the scheduled appointment, and ii) a target area in the healthcare facility to which the patient is to navigate for the scheduled appointment; continuously track a position of the patient within the healthcare facility using the wearable device, in part to determine when the patient has exited the healthcare facility; and update a status of the patient in a digital patient tracker based on the tracked position of the patient within the healthcare facility.

Another implementation of the present disclosure is a method of patient management in a healthcare facility, the method including: determining that a patient is within a threshold distance of the healthcare facility based on geolocation data for a wearable device associated with the patient, wherein the patient has a scheduled appointment with a healthcare provider at the healthcare facility; automatically checking-in the patient for the scheduled appointment once it is determined that the patient is within the threshold distance of the healthcare facility; transmitting, to the wearable device associated with the patient, a first notification indicating: i) that the patient ready to be seen the healthcare provider for the scheduled appointment, and ii) a target area in the healthcare facility to which the patient is to navigate for the scheduled appointment; continuously tracking a position of the patient within the healthcare facility using the wearable device, in part to determine when the patient has exited the healthcare facility; and updating a status of the patient in a digital patient tracker based on the tracked position of the patient within the healthcare facility.

Yet another implementation of the present disclosure is a non-transitory computer-readable medium having instructions stored thereon that, when executed by one or more processors, cause a computing device to: determine that a patient is within a threshold distance of a healthcare facility based on geolocation data for a wearable device associated with the patient, wherein the patient has a scheduled appointment with a healthcare provider at the healthcare facility; automatically check-in the patient for the scheduled appointment once it is determined that the patient is within the threshold distance of the healthcare facility; transmit, to the wearable device associated with the patient, a first notification indicating: i) that the patient ready to be seen the healthcare provider for the scheduled appointment, and ii) a target area in the healthcare facility to which the patient is to navigate for the scheduled appointment; continuously track a position of the patient within the healthcare facility using the wearable device, in part to determine when the patient has exited the healthcare facility; and update a status of the patient in a digital patient tracker based on the tracked position of the patient within the healthcare facility.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a diagram of an example check-in process using the patient management system disclosed herein, according to some implementations.

FIG. 2 is a sequence diagram of communications between the components of the patient management system disclosed herein, according to some implementations.

FIG. 3 is a block diagram of a patient management system, according to some implementations.

FIG. 4 is a flow chart of a process for multimodal patient check-in and management, according to some implementations.

FIG. 5 is a flow chart of a process for multimodal patient check-in and management via a wearable device, according to some implementations.

FIG. 6A-6D are example user interfaces presented on a user device, according to some implementations.

FIG. 7 is an example user interface for a patient management system, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a system and methods for patient management in a healthcare setting are shown, according to various implementations. In particular, the disclosed system and methods incorporate a multimodal technique of managing patient interactions that addresses various shortcomings of current patient check-in and tracking systems. Notably, the disclosed system and methods leverage wearable technology (e.g., smartwatches) to provide users (e.g., patients) with an intuitive and convenient option for checking in to appointments, navigating through a healthcare facility, communicating with healthcare professionals, and more. From the perspective of the healthcare provider/facility, the disclosed system and methods can help to facilitate patient management, reduce congestion, and improve patient experiences.

A central aspect of the disclosed system and methods is the ability to track a location of a patient with respect to a healthcare facility and/or a position of the patient within the healthcare facility using a wearable device associated with the patient. Using location and/or positioning data, herein collectively referred to as “tracking data,” the patient can be automatically checked-in for scheduled appointments and the patient can be managed once checked in. For example, tracking data can be used to determine how many patients are in the healthcare facility, where the patients are physically located in the healthcare facility, and the like. In addition, the wearable device described herein allows a patient to communicate with the healthcare facility and/or their healthcare provider by receiving notifications (e.g., appointment updates, room assignments, etc.) from healthcare facility/provider and/or sending messages (e.g., status updates, location information, etc.) to healthcare facility/provider.

### Example Check-In Process

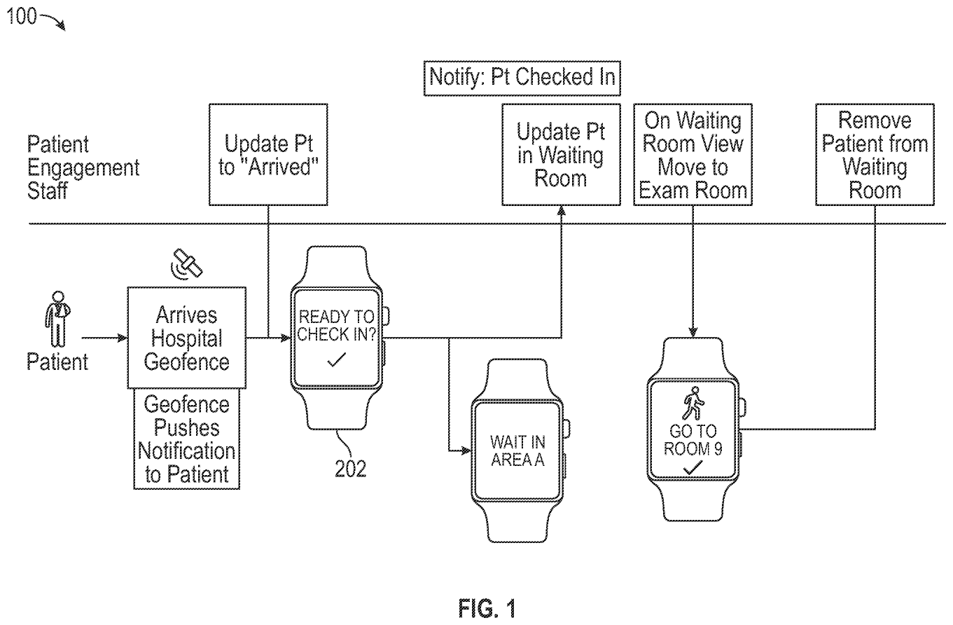

To better explain certain features of the present disclosure from a high level, reference is now made to FIG. 1 in which a diagram of an example check-in process 100 using the disclosed system and methods is shown, according to some implementations. In this example, a patient is initially shown as arriving at a hospital (e.g., a healthcare facility) for an appointment. The patient's arrival is detected through the use of a geofence associated with the hospital. The patient, in this example, carries a wearable device 202 which, in part, is used to determine the patient's geophysical location. In this regard, the patient's geophysical location can be assumed to be the same as the location of wearable device 202 (e.g., since wearable device 202 is worn or carried by the patient), which can be determined (e.g., calculated) using one or more known location/position detection techniques, as described below. The patient's geophysical location is in turn used to determine whether the patient is within the geofence. It should be understood that the patient is the user of wearable device 202; thus, the terms “patient” and “user” may be referenced interchangeably herein.

As known to those in the art, a geofence is a virtual boundary that defines a perimeter around a physical location. Geofences may be defined by specific coordinates or a radius around the physical location, and therefore can be as simple as a circular area defined by a center point and a radius or more complex, encompassing irregular shapes, e.g., using coordinates. For example, the geofence placed around the hospital may be defined by a threshold distance from a center point positioned (e.g., virtually) on the hospital, selected coordinates around the hospital area, streets or addresses around the hospital, etc. A person is considered to “enter” a geofence when their location is detected within the geofenced area and/or once it is detected that they have crossed the virtual boundary that defines the geofence.

As described in greater detail below, wearable device 202 is generally a computing device configured to be worn by a user, e.g., on the wrist, neck, etc. One common example of such a wearable device is a smartwatch; however, other types of wearable devices are contemplated herein. For example, in some cases, wearable device 202 is a smartwatch that is owned by the user/wearer. In any case, wearable device 202 can include a transceiver and/or one or more other components for determining its geophysical location. For example, in some implementations, wearable device 202 includes a satellite transceiver and/or cellular transceiver for location detection via global navigation satellite system (GNSS), global positioning system (GPS), or assisted GNSS/GPS (A-GNSS or A-GPS). Typically, wearable device 202 uses its internal transceiver(s) to detect its location, which is then reported (e.g., wirelessly transmitted) to a remote device (e.g., a server).

In this regard, wearable device 202 may be configured to execute a software program—sometimes referred to as an “application” or “app”—which locally detects the current location of wearable device 202 (e.g., using A-GNSS data, A-GPS data, etc.) and transmits an indication of the detected location to the remote device. For example, wearable device 202 may execute a smartwatch “app” that periodically or continuously transmits location data to a backend server for tracking the location of an associated user. In this manner, the remote device (e.g., server) can automatically identify a patient associated with wearable device 202 based on information that is included in the transmitted location data, such as a device identifier (e.g., an IP address, device IMIE or serial number, a phone number associated with the device, etc.). Alternatively, in implementations where wearable device 202 communicates directly with a healthcare facility's computing system, the location data transmitted by wearable device 202 may include other identifying information. In some implementations, the identifying information and/or location data is encrypted by wearable device 202 prior to transmission. Additional discussion of detecting a patient's location via wearable device 202 is provided below.

It should also be appreciated that, in some implementations, the patient establishes consent for tracking, etc., prior to using the software application. For example, the first time the patient uses the software application, they may be prompted to create an account and/or log in to an existing account, e.g., associated with their healthcare provider. During registration for an account and/or upon logging in to an existing account for the first time, the patient may be prompted to accept terms and conditions that authorize the use of location data for wearable device 202 in determining the patient's location, e.g., for real-time tracking, automatic check-ins, etc. The patient may consent by accepting the terms and conditions with a check box or button, or by providing verbal consent, using facial recognition, etc.

Once the patient is detected within the hospital's geofence (e.g., based on location data of wearable device 202), the hospital is notified of the patient's arrival. In some implementations, a notification is presented to an operator of the hospital's computer system. For example, a notification indicating that the patient has arrived may be presented to hospital staff at a check-in desk. In this manner, the hospital staff can manually add the patient to a digital queue or otherwise update a patient management system to indicate that the patient is on the premises. In other implementations, a patient management system (e.g., associated with and/or operated by the hospital) is automatically updated to add the patient to a queue and/or to reflect the patient's status as “at the hospital” but not checked in. As mentioned above, it should be appreciated that communications from wearable device 202 to the hospital's computing system may be facilitated by a remote (e.g., intermediary) computing device; thus, in some implementations, the hospital is notified by the remote computing device. In other implementations, wearable device 202 may provide a notification directly to the hospital's computing system.

After the patient is determined to be within a threshold distance of the hospital (e.g., within the geofence) and/or after the hospital is notified of the patient's arrival, wearable device 202 may display a prompt to the patient to initiate check-in. In the example shown, the prompt is displayed via a screen of wearable device 202 and asks, “Ready to Check In?”. In some implementations, the patient can accept the prompt to initiate a check-in procedure by tapping an icon or button on the screen of wearable device 202. In some other implementations, the patient may be automatically checked-in, in which case a prompt may not be presented and/or the patient does not need to manually accept the prompt. In any case, a notification may be transmitted to the hospital's computing system to initiate the check-in procedure. In some implementations, the check-in procedure is performed manually, e.g., by hospital staff, after receiving the notification. In other implementations, the notification causes the hospital computing system to automatically perform a predefined check-in procedure.

It should be understood that various healthcare providers may have different procedures and/or requirements for patient check-in; thus, the check-in procedure automatically initiated responsive to the patient's approval of the check-in prompt or based on the patient's location may not be the same between different healthcare providers. For example, patient check-in procedures can include verifying a patient's personal information (e.g., address, phone number, etc.), insurance information, and the like. However, patient check-in generally includes at least indicating a patient status in a patient management system and/or adding the patient to a digital queue. As mentioned herein, a “patient management system” generally refers to a system or software program utilized by a healthcare provider to manage patients, including checking-in patients, tracking assigned room numbers, maintaining schedules, booking appointments, etc. Further details are discussed below.

While described thus far as a device that is owned or otherwise maintained by a single user (e.g., a smartwatch), it should be appreciated that wearable device 202 may be owned or otherwise maintained by another entity and therefore may be permanently or temporarily assigned to a patient, e.g., for the course of a visit to a healthcare facility. For example, a healthcare facility may maintain a number of similar or identical wearable devices (e.g., multiple of wearable device 202) which can be “loaned” to patients for the course of their visit. In this manner, patients can benefit from the various features of wearable device 202 described herein even if they do not own a smartwatch or other similar device. Likewise, the healthcare facility/provider can benefit from patient tracking and other features described herein for patients that do not own their own device. In some such implementations, a patient may be “loaned” wearable device 202 once they arrive at the healthcare facility and/or after checking in for an appointment. In other words, the patient may be allowed to temporarily use wearable device 202 while they are at the healthcare facility.

In the example shown, after the patient accepts the check-in prompt and/or is otherwise checked in, the patient's status in a patient management system is updated to “Pt in Waiting Room.” In other words, the patient is indicated as being in the waiting room of the hospital and/or is added to a digital queue prior to their appointment with a provider. In conjunction, wearable device 202 may display a notification prompting the patient to move to a specific area of the hospital (e.g., “Waiting Area A”) and/or otherwise indicating the status of the patient's appointment. In some implementations, the notification is provided by the hospital's computing system (e.g., directly or through an intermediary computing device). For example, the patient may be manually or automatically assigned to a waiting room (e.g., based on hospital capacity, the position of other patients, etc.) and an indication of the assigned waiting room may be transmitted to wearable device 202, which causes wearable device 202 to display a notification.

Once the patient is ready to be seen by a healthcare professional, wearable device 202 may be updated with an indication that the patient is ready to be seen and/or with a location of the patient's appointment. In the example shown, wearable device 202 displays “Go to Room 9,” prompting the patient to navigate to a target area of the hospital. In some implementations, a notification is provided by the hospital's computing system with the assigned room number. For example, the patient may be automatically or manually assigned to an examination room via the patient management system, and, in response, a notification may be transmitted to wearable device 202. In some implementations, the notification prompting the patient to navigate to a target area of the hospital includes an icon or button that can be selected by the patient to acknowledge the prompt, which in turn can indicate that the patient is beginning to navigate to the target area. In some implementations, the patient's status or location in the patient management system can be updated to remove the patient from a digital “waiting room” or to otherwise remove the patient from a digital queue.

While FIG. 1 illustrates only check-in process 100, it should be appreciated that the disclosed system and methods provide a number of additional features not already mentioned. In some implementations, wearable device 202 can facilitate communications between the patient and hospital staff (e.g., via the hospital's computing system and/or patient management system). For example, the patient can use wearable device 202 to ask questions, update their status or location, and the like, and can receive updates/notifications from the hospital (e.g., emergency alerts, appointment updates, etc.). In some implementations, wearable device 202 can facilitate payment of out-of-pocket expenses, such as copays, related to the patient's appointment. In some implementations, wearable device 202 can provide real-time directions (e.g., via augmented reality (AR)) to the patient for navigating through a healthcare facility. These and other features are described in greater detail below.

### Patient Management System

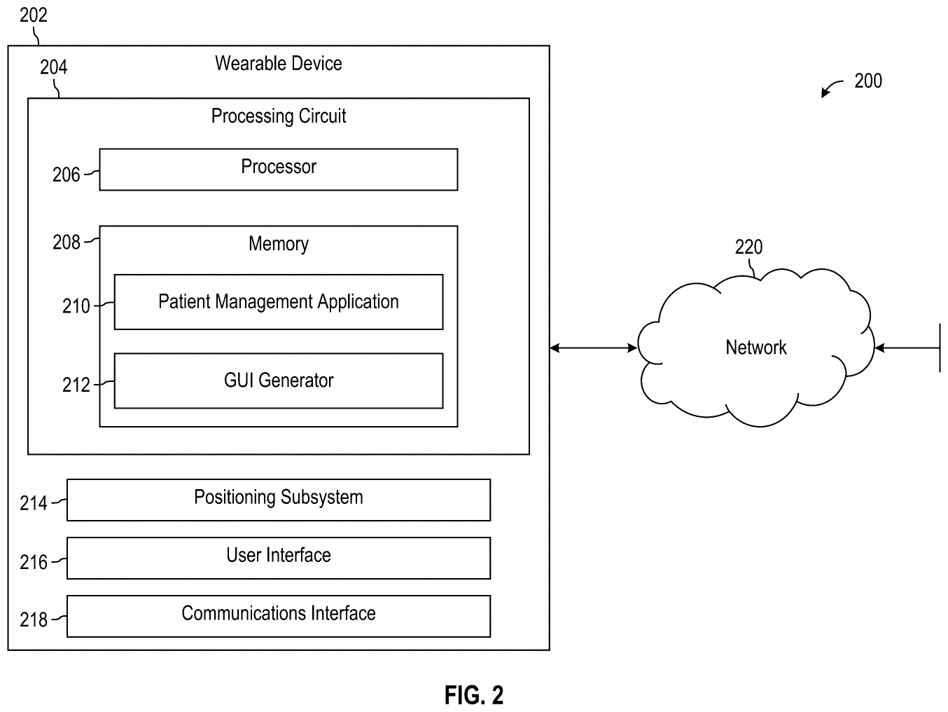

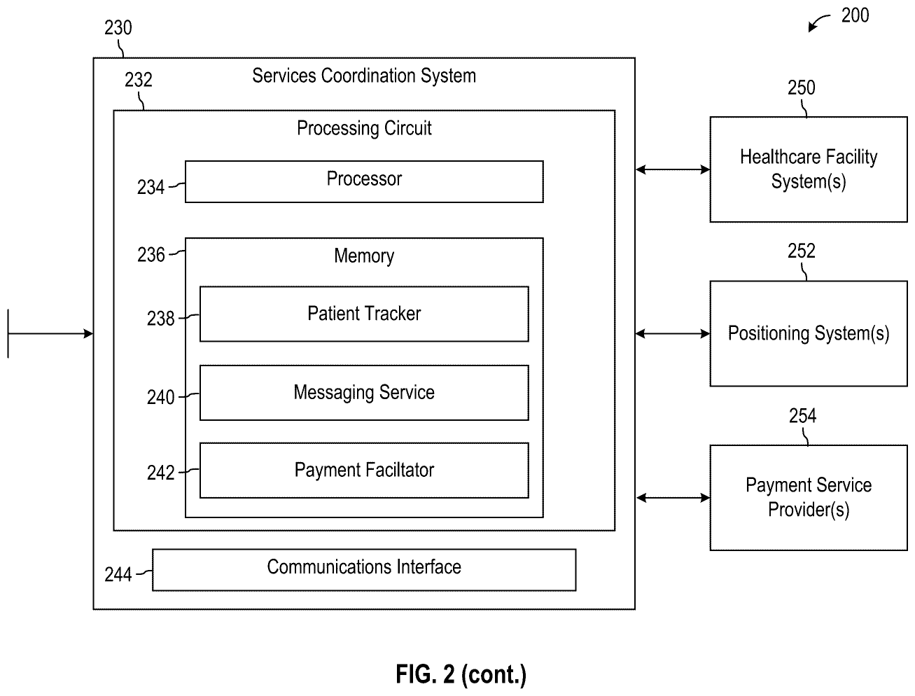

Referring now to FIG. 2, a detailed block diagram of a patient management system 200 is shown, according to some implementations. Mainly, patient management system 200 is shown to include wearable device 202 and a services coordination system 230, communicably coupled via a network 220. As discussed above, wearable device 202 is a computing device configured to be worn by a user, such as a smartwatch. Services coordination system 230 is a computing system that is separate (e.g., remote) from wearable device 202 and that handles services such as patient tracking, emergency alert coordination, payment facilitation, and the like, as discussed in greater detail below. In some implementations, for example, services coordination system 230 is a back-end server (e.g., a cloud server). While only one of wearable device 202 is shown for the sake of brevity, patient management system 200 can include any number of wearable devices; therefore, it should be understood that services coordination system 230 can interact with any number of wearable devices as described herein. Network 220 is any suitable type of network that allows data to be communicated between (e.g., transmitted from/to) wearable device 202 and services coordination system 230. For example, network 220 may be a wide area network (WAN), such as the Internet, a local area network (LAN), a virtual private network (VPN), a cellular network, or the like; however, it should be appreciated that these examples are not intended to be limiting.

Wearable device 202 is shown to include a processing circuit 204, which further includes a processor 206 and memory 208. Processor 206 can be a general-purpose processor, an application specific integrated circuit (ASIC), one or more field programmable gate arrays (FPGAs), a group of processing components (e.g., a central processing unit (CPU)), or other suitable electronic processing structures. In some embodiments, processor 206 is configured to execute program code stored on memory 208 to cause wearable device 202 to perform one or more operations, as described below in greater detail. It will be appreciated that, in embodiments where wearable device 202 is part of another computing device, the components of wearable device 202 may be shared with, or the same as, the host device. For example, if wearable device 202 is implemented via a smartwatch or smartphone, then wearable device 202 may utilize the processing circuit, processor(s), and/or memory of the smartwatch/smartphone to perform the functions described herein.

Memory 208 can include one or more devices (e.g., memory units, memory devices, storage devices, etc.) for storing data and/or computer code for completing and/or facilitating the various processes described in the present disclosure. In some embodiments, memory 208 includes tangible (e.g., non-transitory), computer-readable media that stores code or instructions executable by processor 206. Tangible, computer-readable media refers to any physical media that is capable of providing data that causes wearable device 202 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 208 can include random access memory (RAM), read-only memory (ROM), erasable programmable read-only memory (EPROM), electronically erasable programmable read-only memory (EEPROM), hard drive storage, temporary storage, non-volatile memory, flash memory, optical memory, or any other suitable memory for storing software objects and/or computer instructions. Memory 208 can include database components, object code components, script components, or any other type of information structure for supporting the various activities and information structures described in the present disclosure. Memory 208 can be communicably connected to processor 206, such as via processing circuit 204, and can include computer code for executing (e.g., by processor 206) one or more processes described herein.

While shown as individual components, it will be appreciated that processor 206 and/or memory 208 can be implemented using a variety of different types and quantities of processors and memory. For example, processor 206 may represent a single processing device or multiple processing devices. Similarly, memory 208 may represent a single memory device or multiple memory devices. Additionally, in some embodiments, wearable device 202 may be implemented within a single computing device (e.g., one server, one housing, etc.). In other embodiments, wearable device 202 may be distributed across multiple servers or computers (e.g., that can exist in distributed locations). For example, wearable device 202 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

Memory 208 is shown to include a patient management application 210 that facilitates various patient management functions described herein. Of note, patient management application 210 facilitates automated patient check-in, patient-provider communications, and indoor navigation (e.g., within a healthcare facility), among other features. In this regard, patient management application 210 may be implemented as a software application that is installed on wearable device 202—also referred to as a “smartwatch app.” In some such implementations, patient management application 210 may be implemented as code stored on memory 208 that can be executed by processor 206 to perform various functions as described herein.

In some implementations, patient management application 210 may be associated with a specific healthcare facility or group of healthcare facilities. For example, patient management application 210 may be associated with a particular healthcare group that operates at multiple locations. In other implementations, patient management application 210 is not associated with a specific healthcare facility or group of healthcare facilities and/or is associated with a plurality of different healthcare facilities/groups. In any case, in some implementations, patient management application 210 may require a user to create an account and/or log in to an existing account to access the features described herein. For example, a user may provide credentials (e.g., email address and password) used to access a website associated with a particular healthcare provider in order to access patient management application 210.

As mentioned, in some implementations, patient management application 210 facilitates an automated check-in process to a healthcare facility for a user (e.g., a patient of the healthcare facility) through a multimodal approach. Initially (e.g., prior to the user arriving at the healthcare facility), patient management application 210 may monitor the user's location to detect when the user is within a threshold distance of the healthcare facility, triggering the automated check-in process. In some implementations, patient management application 210 collects location data via a positioning subsystem 214, described in greater detail below. Positioning data generally includes coordinates of the user, e.g., based on a location of wearable device 202, but can also include other formats of location data (e.g., a street address).

Patient management application 210 may periodically or continuously collect location data via positioning subsystem 214 and can compare it to a known location of the healthcare facility—or, more specifically, a geofence associated with the healthcare facility—to determine if the user is within the threshold distance. Additionally, or alternatively, patient management application 210 may transmit location data to services coordination system 230, in which case services coordination system 230 may be configured to determine if the user is within the geofence associated with the healthcare facility (e.g., which reduces computational requirements for wearable device 202 by offloading processing). It should be appreciated that, in some implementations, patient management application 210 collects location data only when active (e.g., only when the application is running/executing) to protect a user's privacy. In other implementations, patient management application 210 can be configured to operate in the background such that the user's location can be tracked even if the application is not in use.

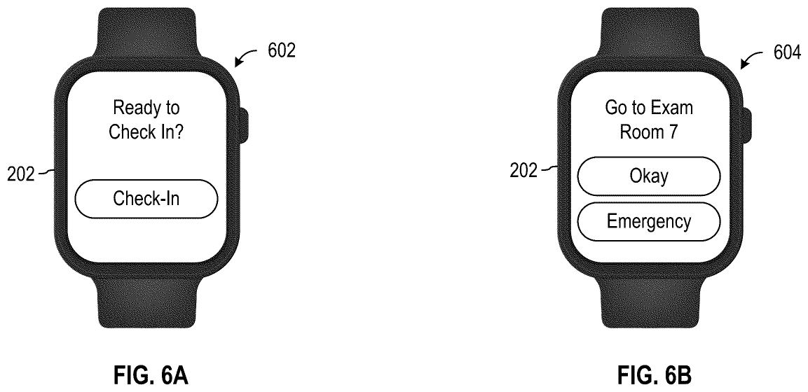

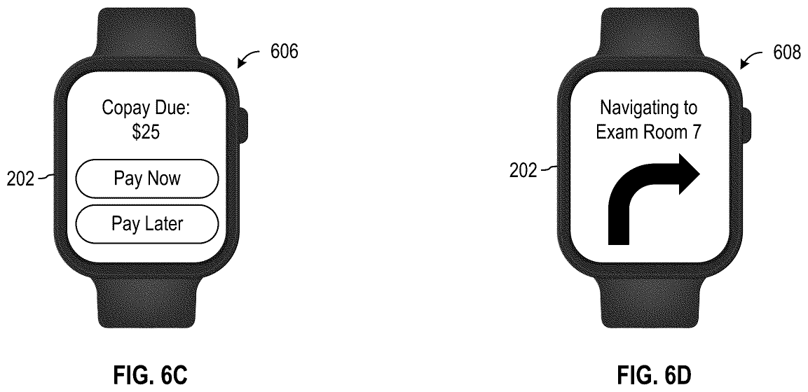

In some implementations, once it is determined that the user (e.g., the wearer of wearable device 202) is within a threshold distance of the healthcare facility, patient management application 210 can transmit a notification to services coordination system 230 indicating as much. Specifically, the notification may indicate that the user is within the geofence associated with the healthcare facility. In some such implementations, receipt of this notification causes services coordination system 230 to initiate the aforementioned automated check-in procedure, which is described in greater detail below. Patient management application 210 may then cause a graphical user interface (GUI) generator 212 to generate a notification for display via a user interface 216, which prompts the user to check-in to the healthcare facility. An example of said notification is shown in FIG. 6A, described below.

GUI generator 212, as mentioned, is configured to generate GUIs for display via user interface 216. A GUI is an interface through which a user interacts with a device (e.g., wearable device 202) that typically contains one or more graphical elements, such as text, images, graphics, icons, buttons, and the like. User interface 216 includes one or more components that facilitate physical user interaction with wearable device 202. In particular, user interface 216 generally includes a screen (e.g., an LED or LCD screen) configured to display the GUIs generated by GUI generator 212. In some implementations, user interface 216 includes a user input component for receiving user inputs. In some such implementations, user interface 216 is a touchscreen display that can both display GUIs and receive user inputs via a touch (e.g., through capacitive touch sensing). User interface 216 can additionally or alternatively include other user interface components, such as buttons, lights (e.g., LEDs), a microphone, a speaker, and the like; the present disclosure is not intended to be limiting in this regard.

In some implementations, automated check-in is available to the user only if they have a scheduled appointment with a healthcare provider. In other implementations, the user may be able to check-in without a scheduled appointment, as long as they are an existing patient of the healthcare facility (e.g., and therefore do not require new patient admissions). In some implementations, the automated check-in process is initiated responsive to a user input via user interface 216; however, in other implementations, the automated check-in process is automatically initiated. In some implementations, as part of the automated check-in process, patient management application 210 can utilize various sensors of wearable device 202 (not shown) to collect biometric data, which can save time during an appointment. For example, wearable device 202 may include sensors for measuring the user's heart rate, blood oxygen concentration, and/or blood pressure, all of which can be recorded and transmitted to services coordination system 230 during check-in.

Once the user of wearable device 202 (e.g., a patient) is checked in, patient management application 210 may cause GUI generator 212 to generate and display a second notification indicating that the user is checked in and/or indicating a first target area for the patient to navigate to (e.g., a waiting room). In some implementations, the second notification is generated responsive to patient management application 210 receiving an indication that the user has been successfully checked in from services coordination system 230. Likewise, in some implementations, services coordination system 230 provides patient management application 210 with an indication of an assigned target area (e.g., a waiting room) which is presented with/in the notification. After some time, when the user is ready to be seen by a healthcare provider, patient management application 210 may receive another notification from services coordination system 230 indicating the user is ready to be seen and/or providing a new target area within the healthcare facility to which the user should navigate. Then, patient management application 210 may cause GUI generator 212 to generate a third notification (e.g., presented via user interface 216) indicating the target area. For example, user interface 216 may display “Proceed to Exam Room A” when the user is ready to be seen. An example of said notification is shown in FIG. 6B, described below.

While the user is navigating through the healthcare facility to a target area (e.g., an exam room), patient management application 210 may be configured to monitor the user's position within the healthcare facility. The user's position within the healthcare facility can be used to manage facility capacities and/or congestion, track patients in case of emergencies, and provide users of wearable device 202 with real-time directions to navigate to target areas. In this regard, patient management application 210 may periodically or continuously report a user's position to services coordination system 230 for patient tracking. Patient management application 210 may also generate directions through the healthcare facility to a target area based on the user's current position. In some implementations, directions are provided in real- or near real-time via user interface 216, as shown in FIG. 6D described below. Generally, patient management application 210 determines a user's position using positioning subsystem 214.

Positioning subsystem 214 generally includes one or more components for detecting one or both of a geographical location (e.g., prior to a user entering a healthcare facility) and an indoor position (e.g., when the user is inside the healthcare facility) of wearable device 202. In this regard, positioning subsystem 214 typically includes one or more transceivers for satellite communications (e.g., for GNSS or GPS) and/or one or more transceivers associated with an indoor positioning system (IPS). For example, positioning subsystem 214 may include at least one satellite transceiver for communicating with a GNSS or GPS satellite, e.g., to determine geographical coordinates of wearable device 202. Positioning subsystem 214 may also include short-range wireless transceiver(s) (e.g., Wi-Fi transceiver) for utilizing an IPS to determine a position when indoors. In some implementations, positioning subsystem 214 includes multiple transceivers to facilitate A-GNSS or A-GPS location tracking. For example, as those in the art will appreciate, A-GPS utilizes both GPS and cellular signals to determine a device's location; thus, positioning subsystem 214 may utilize a satellite transceiver and a cellular transceiver for detecting location when outside a healthcare facility and may utilize a Wi-Fi transceiver for indoor position detection.

In some implementations, patient management application 210 can prompt a user to make a payment for any out-of-pocket expenses related to an appointment and, optionally, can facilitate said payment. For example, after a patient is checked-in for an appointment, patient management application 210 may cause GUI generator 212 to generate and present (e.g., via user interface 216) a GUI indicating to the user a copay amount for the appointment and/or prompting the user to submit payment for a copay. An example of such a GUI is shown in FIG. 6C, described below. In some such implementations, patient management application 210 receives information about what a user owes for an appointment from services coordination system 230. Alternatively, patient management application 210 simply receives an indication that the user is subject to a copay or other payment without an indication of a specific amount. In either case, patient management application 210 may facilitate payment for out-of-pocket expenses by communicating with services coordination system 230 or an outside payment facilitator. For example, patient management application 210 may transmit an indication of user approval for the out-of-pocket expense payment, causing services coordination system 230 or another payment facilitator to send a payment to the payee (e.g., a healthcare provider). In another example, patient management application 210 may electronically transfer funds from a source (e.g., a mobile wallet) to the payee. Other payment facilitation techniques are described below.

Even more generally, patient management application 210 can facilitate messaging between a patient (e.g., the user of wearable device 202) and a healthcare facility/provider. Various notifications are described above, for example, and shown in FIGS. 6A-6D, including notifications for checking-in to an appointment, navigating through a healthcare facility, and making payments for out-of-pocket expenses. Patient management application 210 can also allow healthcare providers and/or other users of services coordination system 230 to transmit other types of notifications to wearable device 202 which cause GUI generator 212 to generate and present GUIs via user interface 216. For example, a healthcare provider can provide updates on wait times, a patient's position in a digital queue, and the like. Patient management application 210 can also allow a user to send messages to a healthcare provider or other recipient. For example, the user may be presented (e.g., via user interface 216) with a variety of predefined messages that can be sent with a few taps. In another example, the user may record and transmit a voice message, e.g., using user interface 216 of wearable device 202. It will be appreciated that the present disclosure is not limited in this regard.

Wearable device 202 is also shown to include a communications interface 218 that facilitates communications between wearable device 202 and any external components or devices, including services coordination system 230. Accordingly, communications interface 218 can be or can include a wired or wireless communications interface (e.g., jacks, antennas, transmitters, receivers, transceivers, wire terminals, etc.) for conducting data communications, or any combination of wired and/or wireless communication interfaces. As noted above, in some implementations, communications via communications interface 218 are conducted via network 220 (e.g., a WAN, the Internet, a cellular network, etc.); however, it should be understood that communications interface 218 may also be configured for direct communications (e.g., local wired or wireless communications). For example, communications interface 218 may include cellular or mobile phone communications transceivers for wireless data communications via a cellular network. In another example, communications interface 218 can include a Wi-Fi transceiver for communicating via a wireless communications network and/or a short-range wireless transceiver for direct, wireless communications (e.g., via Bluetooth©). With reference to positioning subsystem 214, above, it should be appreciated that a Wi-Fi and/or short-range wireless transceiver can also be used for indoor positioning via an IPS. In yet another example, communications interface 218 may include one or more universal serial bus (USB) ports for communicably coupling wearable device 202 to another device.

Still referring to FIG. 2, services coordination system 230 is also shown to include a processing circuit 232 which, like the processing circuit of wearable device 202, includes a processor 234 and memory 236. Processor 234 can be a general-purpose processor, an ASIC, one or more FPGAs, a group of processing components (e.g., a CPU), or other suitable electronic processing structures. In some embodiments, processor 234 is configured to execute program code stored on memory 236 to cause services coordination system 230 to perform one or more operations, as described below in greater detail. It will be appreciated that, in embodiments where services coordination system 230 is part of another computing device, the components of services coordination system 230 may be shared with, or the same as, the host device. For example, if services coordination system 230 is implemented via a server (e.g., a cloud server), then services coordination system 230 may utilize the processing circuit, processor(s), and/or memory of the server to perform the functions described herein.

Memory 236 can include one or more devices (e.g., memory units, memory devices, storage devices, etc.) for storing data and/or computer code for completing and/or facilitating the various processes described in the present disclosure. In some embodiments, memory 236 includes tangible (e.g., non-transitory), computer-readable media that stores code or instructions executable by processor 234. Tangible, computer-readable media refers to any physical media that is capable of providing data that causes services coordination system 230 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 236 can include RAM, ROM, EPROM, EEPROM, hard drive storage, temporary storage, non-volatile memory, flash memory, optical memory, or any other suitable memory for storing software objects and/or computer instructions. Memory 236 can include database components, object code components, script components, or any other type of information structure for supporting the various activities and information structures described in the present disclosure. Memory 236 can be communicably connected to processor 234, such as via processing circuit 232, and can include computer code for executing (e.g., by processor 234) one or more processes described herein.

While shown as individual components, it will be appreciated that processor 234 and/or memory 236 can be implemented using a variety of different types and quantities of processors and memory. For example, processor 234 may represent a single processing device or multiple processing devices. Similarly, memory 236 may represent a single memory device or multiple memory devices. Additionally, in some embodiments, services coordination system 230 may be implemented within a single computing device (e.g., one server, one housing, etc.). In other embodiments, services coordination system 230 may be distributed across multiple servers or computers (e.g., that can exist in distributed locations). For example, services coordination system 230 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

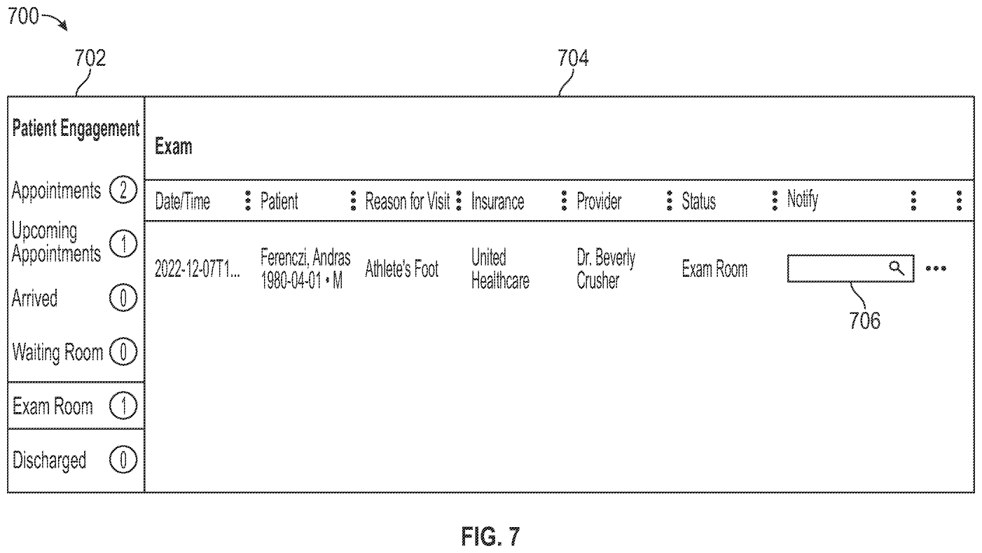

Memory 236 is shown to include a patient tracker 238 configured to track patient statuses and/or locations (e.g., within a healthcare facility). In some implementations, patient tracker 238 can determine when a patient is within a healthcare facility's geofence based on data received from wearable device 202. Specifically, in some such implementations, wearable device 202 can transmit location data (e.g., coordinates) to patient tracker 238 such that patient tracker 238 can track the wearer's location. In other such implementations, wearable device 202 can transmit a notification to patient tracker 238 when it determines that it is within the healthcare facility's geofence. In either case, patient tracker 238 can update a patient location and/or status accordingly, e.g., to indicate that the patient is at the healthcare facility but not checked in. An example patient tracking interface is shown in FIG. 7, described below. It should be appreciated that various methods of tracking patient locations and statuses are contemplated herein. For example, patient tracker 238 may maintain a table or database of patients that include a field for a patient status and/or location. In another example, patient tracker 238 may update electronic patient records to reflect statuses/locations. The present disclosure is not intended to be limiting in this regard.

To this point, in some implementations, patient tracker 238 can facilitate automated patient check-in directly and/or by communicating with a healthcare facility system 250. Specifically, patient tracker 238 may be configured to perform various automatic check-in processes (e.g., as discussed above) responsive to detecting that a patient is near a healthcare facility. For example, patient tracker 238 may retrieve and/or verify patient information, check that the patient has a scheduled appointment, etc., and, once complete, can update a status of the patient to “checked in.” Additionally, or alternatively, patient tracker 238 may transmit a notification to healthcare facility system 250 indicating that the patient is near the healthcare facility and ready to be checked in. In some such implementations, healthcare facility system 250 itself may facilitate check-in of the patient and may report back to patient tracker 238 once the patient has been checked in—in which case patient tracker 238 may update the patient's status accordingly.

As referenced herein, healthcare facility system 250 generally includes one or more computing systems/devices associated with and/or operated by a healthcare facility. For example, healthcare facility system 250 may include a server or multiple servers associated with and/or maintained by a healthcare facility, e.g., to manage patient records and appointments, etc. In another example, healthcare facility system 250 may include one or more desktop computers or workstations at a healthcare facility (e.g., a computer at a check-in desk). While shown in communication with services coordination system 230, it should be appreciated that healthcare facility system 250 may also/alternatively be in combination with wearable device 202 or, more generally, can be remotely accessed via network 220. It should also be appreciated that, in some implementations, various functions of services coordination system 230 may be implemented by or hosted on healthcare facility system 250. In other words, services coordination system 230 may be a component of healthcare facility system 250, in some cases, and/or various functions described herein with respect to services coordination system 230 may be at least partially implemented by healthcare facility system 250, or vice versa. For example, services coordination system 230 may be part of healthcare facility system 250—and therefore operated by a healthcare facility—as opposed to being hosted on a separate device (e.g., a server). However, the present disclosure is not intended to be limiting in this regard.

In addition to maintaining patient statuses and determining when a patient has arrived at a healthcare facility (e.g., for check-in), patient tracker 238 may be configured to track a patient's position within the healthcare facility. In some implementations, a patient's position is simply updated manually or automatically based on the patient's status. For example, as discussed in greater detail below, a patient's position may be updated to a specific room number once they are assigned to an examination room for an appointment. In some implementations, however, patient tracker 238 can track a patient's position in real- or near-real time by receiving position data from wearable device 202 (e.g., as the user moves through the healthcare facility) and/or by communicating with a position system 252. As mentioned above, for example, wearable device 202 may utilize an IPS associated with the healthcare facility for indoor navigation and position detection, in which case wearable device 202 may regularly or continuously transmit indoor position data to patient tracker 238. In other implementations, patient tracker 238 uses position system 252 to actively track the patient's movements.

Positioning system 252, as mentioned herein, generally includes one or more external systems for detecting the location and/or position of a patient, e.g., through wearable device 202. In some implementations, position system 252 includes an IPS associated with and/or installed in a healthcare facility. For example, position system 252 may include a number of IPS beacons (e.g., Wi-Fi transceivers) positioned throughout the healthcare facility which can be utilized by wearable device 202 to determine a position within the healthcare facility. In some implementations, position system 252 includes a satellite navigation system (e.g., GNSS, GPS) which wearable device 202 utilizes for geographical locating. In some implementations, position system 252 itself may be configured to determine a location of wearable device 202 (e.g., as opposed to wearable device 202 detecting its own location), which can be communicated to services coordination system 230. However, wearable device 202 is often configured to utilize position system 252 to detect its own location/position, which is communicated to services coordination system 230. While shown in communication with services coordination system 230, it should be appreciated that position system 252 may also/alternatively be in combination with wearable device 202 or, more generally, can be remotely accessed via network 220.

Memory 236 is also shown to include a messaging service 240 that facilitates messaging between wearable device 202 and a healthcare facility/provider. In particular, messaging service 240 can transmit notifications to wearable device 202 which, as discussed above, cause wearable device 202 to generate and/or display GUIs to a user. Likewise, messaging service 240 can receive communications from wearable device 202 which are passed to a healthcare provider (e.g., transmitted to healthcare facility system 250) and/or cause services coordination system 230 to initiate various actions described herein. As mentioned above, some of the various notifications that can be presented to a user of wearable device 202—and thus which can be transmitted to wearable device 202 by messaging service 240—include notifications prompting the user to check-in for an appointment, notifications prompting the user to pay for out-of-pocket expenses, notifications prompting the user to navigate to specific areas in the healthcare facility (e.g., exam rooms), and the like.

In addition, messaging service 240 may be configured to transmit emergency alerts to wearable device 202. For example, in the event of a fire, messaging service 240 may transmit an alert to wearable device 202, causing wearable device 202 to present a GUI that prompt the user to evacuate the building and/or navigate to a designated area outside the building. In this regard, services coordination system 230 may interface with an emergency alert system (e.g., one of healthcare facility system 250) for the healthcare facility and/or may receive emergency alerts from healthcare facility system 250. With respect to emergency alerts, it should also be appreciated that patient tracker 238 can assist with emergency response by maintaining up-to-date positioning and/or status information for all patients in the healthcare facility. For example, patient tracker 238 can track the number of patients in the healthcare facility and where they are located.

As mentioned, messaging service 240 can also facilitate messaging from a patient (e.g., a user of wearable device 202) to a healthcare provider. In some implementations, messaging service 240 can receive messages from wearable device 202 which are simply passed to the healthcare provider. For example, messages may be transmitted to healthcare facility system 250. In some implementations, messaging service 240 can store contact information for individual staff members and/or healthcare professionals at a healthcare facility such that a patient can send a message to an individual staff member or healthcare professional via messaging service 240. For example, messaging service 240 may route/forward messages accordingly. In some implementations, messaging service 240 can process patient messages in order to update patient tracker 238 and/or the patient's electronic record. For example, in response to approval for automated check-in received from wearable device 202 (e.g., responsive to a prompt for the user to check-in), messaging service 240 may cause patient tracker 238 to update the patient status and/or may communicate with healthcare facility system 250 to initiate an automated check-in procedure.

In some implementations, memory 236 includes a payment facilitator 242 that facilitates payments for various expenses between a patient and a healthcare provider. In particular, payment facilitator 242 may facilitate payment for out-of-pocket expenses related to a patient's appointment with a healthcare provider, e.g., responsive to a prompt via wearable device 202. In some implementations, payment facilitator 242 is configured to determine whether a patient owes out-of-pocket expenses, such as a copay, by communicating with healthcare facility system 250. For example, responsive to providing healthcare facility system 250 with an indication that a patient is ready to be checked in and/or has checked in, healthcare facility system 250 may provide services coordination system 230 with an indication that the patient owes a copay. Optionally, healthcare facility system 250 may indicate the amount of the copay as well. Payment facilitator 242 may then communicate with wearable device 202 to cause wearable device 202 to prompt the user to submit payment for the expenses. In some implementations, rather than communicating with healthcare facility system 250 or other external device, payment facilitator 242 itself may determine whether the patient's appointment requires a copay.

In any case, payment facilitator 242 may be configured to facilitate an electronic funds transfer to/from a payment service provider 254. Specifically, payment facilitator 242 may coordinate the transfer of funds between two different payment service providers (e.g., one associated with each of the patient and the healthcare provider), from the patient to a service provider, or from a service provider to a healthcare provider. For example, payment facilitator 242 may electronically transfer funds from a digital wallet associated with a user of wearable device 202 to a healthcare provider's bank account. While shown in communication with services coordination system 230, it should be appreciated that payment service provider 254 may also/alternatively be in combination with wearable device 202 or, more generally, can be remotely accessed via network 220.

Services coordination system 230 is also shown to include a communications interface 244 that facilitates communications between services coordination system 230 and any external components or devices, including wearable device 202. Accordingly, communications interface 244 can be or can include a wired or wireless communications interface (e.g., jacks, antennas, transmitters, receivers, transceivers, wire terminals, etc.) for conducting data communications, or any combination of wired and wireless communication interfaces. In some embodiments, communications via communications interface 244 are direct (e.g., local wired or wireless communications) or via network 220 (e.g., a WAN, the Internet, a cellular network, etc.) as described above. For example, communications interface 244 may include one or more Ethernet ports for communicably coupling services coordination system 230 to network 220 (e.g., the Internet). In another example, communications interface 244 can include a Wi-Fi transceiver for communicating via a wireless communications network.

While not explicitly shown in FIG. 2, it should be appreciated that communications between components of patient management system 200 may be facilitated by respective application programming interfaces (APIs). Specifically, patient management application 210 and/or services coordination system 230 may include respective APIs that facilitate communications with outside devices. For example, patient management application 210 may remotely access and/or communicate with services coordination system 230 via a “service coordination system API”. More broadly, each of patient management application 210 and services coordination system 230 may implement their own respective APIs. In other words, patient management application 210 and services coordination system 230 may interactive via a “patient management application API” and a “services coordination system API,” respectively. Suitable API protocols include, but are not limited to, REST, SOAP, RPC, and the like.

### Example Appointment

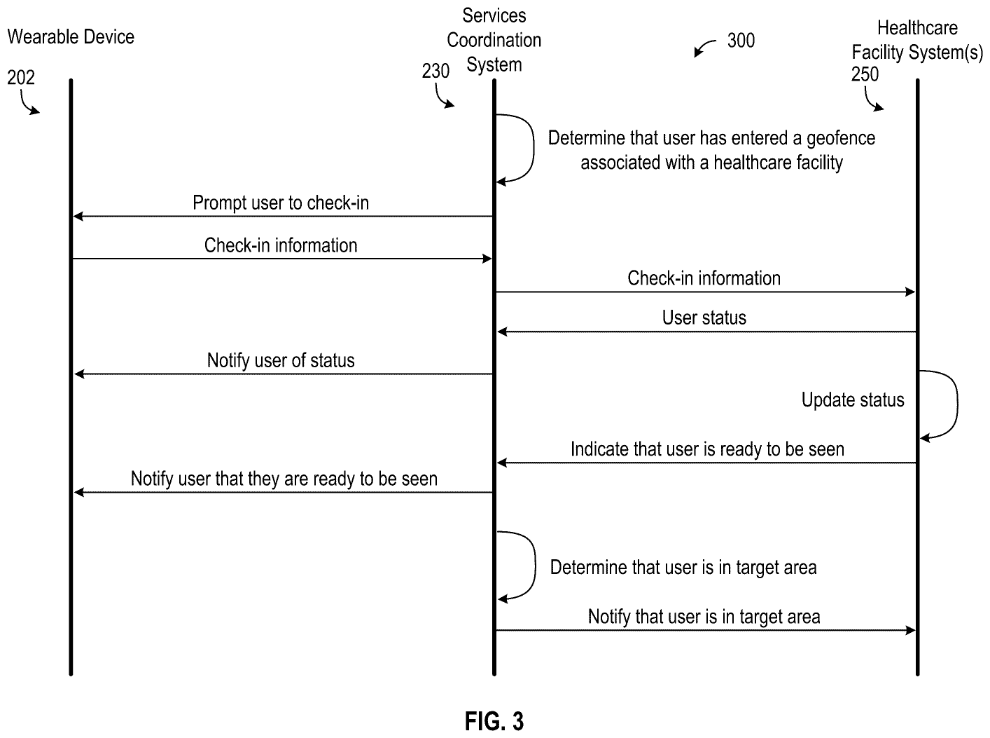

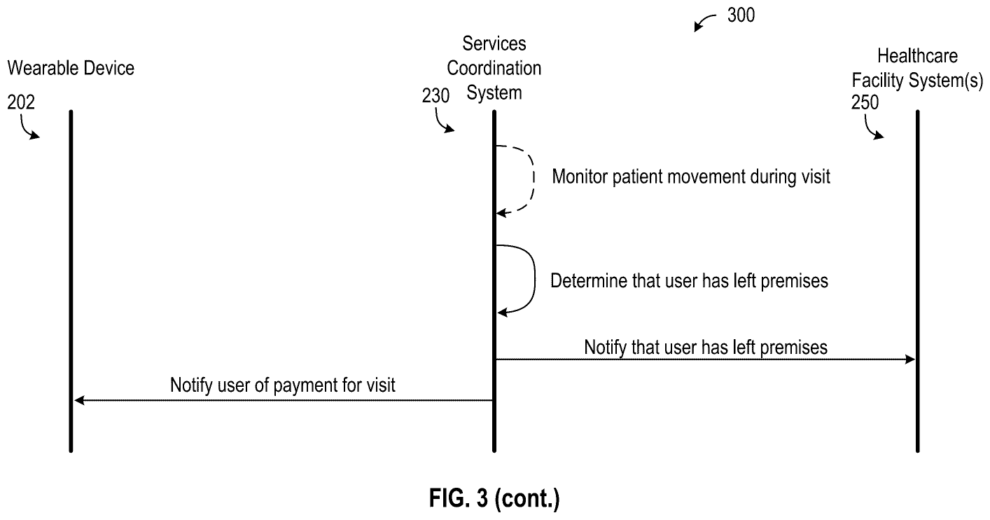

Referring now to FIG. 3, an example sequence diagram 300 of communications between the components of patient management system 200 is shown, according to some implementations. In this example, a patient is visiting a healthcare facility for a scheduled appointment and is carrying wearable device 202 (e.g., a smartwatch) before arriving at the healthcare facility. Sequence diagram 300 therefore illustrates communications between wearable device 202, services coordination system 230, and healthcare facility system(s) 250 over an example patient visit to a healthcare facility, accordingly to at least one implementation of patient management system 200 as discussed above. In this regard, the following description of sequence diagram 300 may be best understood in conjunction with the preceding description of FIGS. 1 and 2. It should also be appreciated that, while reference is made to healthcare facility system 250, the various functionalities thereof (e.g., described below) may be performed by services coordination system 230, in some implementations.

Initially, services coordination system 230 determines that wearable device 202 has entered a geofence associated with a healthcare facility. As mentioned above, services coordination system 230 may make this determination based on location data or a notification received from wearable device 202. Services coordination system 230 then transmits a notification to wearable device 202 for display to a user, prompting the user to check-in for an appointment. In some such implementations, services coordination system 230 may first check whether the user has a scheduled appointment, e.g., by communicating with healthcare facility system 250. Alternatively, services coordination system 230 may transmit a notification to healthcare facility system 250 when wearable device 202 is detected within the geofence, in which case healthcare facility system 250 may check if the user has a scheduled appointment and/or may prompt the user to check in (e.g., by transmitting a notification via services coordination system 230 or causing services coordination system 230 to send the notification).

Responsive to the prompt to check-in, the user of wearable device 202 may provide an input (e.g., tapping “yes” on a GUI) and an indication of the user input (e.g., check-in information) may be transmitted to services coordination system 230. In some implementations, the indication is simply the user response to the prompt (e.g., a “yes” or “no”); however, in other implementations, various check-in related information may be transmitted. For example, wearable device 202 may store or retrieve information about the user (e.g., the user's name, address, insurance information, etc.), which is transmitted to services coordination system 230 responsive to the check-in prompt. Services coordination system 230 may then inform healthcare facility system 250 that the user has checked in by transmitting a notification and/or forwarding the check-in information provided by wearable device 202. In some implementations, services coordination system 230 may also update a status and/or location of the user—herein referred to as a patient—in a patient tracker (e.g., patient tracker 238). Alternatively, or additionally, healthcare facility system 250 may update the patient's status and/or location in its own patient tracking system. In some implementations, services coordination system 230 or healthcare facility system 250 add the patient to a digital queue.

After the patient is checked in, healthcare facility system 250 may provide an indication of the patient's status to services coordination system 230, which may also be forwarded to wearable device 202 for presentation to the patient. For example, healthcare facility system 250 may indicate that the patient is “checked in” and/or may indicate an assigned waiting area in the healthcare facility. In another example, healthcare facility system 250 may indicate to the patient their position in a digital queue. Alternatively, services coordination system 230 may track the patient's status and provide an indication to wearable device 202. It should be understood that, in some implementations, services coordination system 230 and/or healthcare facility system 250 may automatically track and/or update the patient's status. For example, services coordination system 230 may update statuses for a number of patients as they work their way through a digital queue. In other implementations, however, patient statuses and/or assigned locations (e.g., waiting rooms, exam rooms, etc.) may be manually entered, e.g., by an employee of the healthcare facility. For example, a staff member that manages a check-in area that the healthcare facility may manually coordinate patient status and locations, which are entered into a user interface of healthcare facility system 250.

At some point, the patient's status is updated to indicate that the patient is ready to be seen by a healthcare professional (e.g., for a scheduled appointment). For example, the patient's status may be periodically or regularly updated (e.g., by a healthcare facility staff member or automatically) until the patient is ready to be seen, or the patient's status may simply remain as “waiting to be seen” until the healthcare professional is ready for the appointment. In any case, once the patient is ready to be seen for their appointment, healthcare facility system 250 and/or services coordination system 230 may transmit a notification to wearable device 202. The notification may indicate to the patient that they are ready to be seen for their appointment and, optionally, may indicate a target area of the healthcare facility for the patient to travel to for the appointment. For example, the patient may be assigned to an examination room once they are ready to be seen such that the room number is transmitted to wearable device 202 with the notification.

The patient's position within the healthcare facility may optionally be tracked as they navigate to the target area (e.g., an assigned examination room), as discussed in great detail below. For example, wearable device 202 may periodically transmit its position to services coordination system 230 for tracking, in which case services coordination system 230 may determine once the patient is within the target area (e.g., the exam room). Alternatively, or additionally, wearable device 202 may track its position locally and may transmit a notification to services coordination system 230 once the patient reaches the target area of the facility. In some implementations, services coordination system 230 transmits a notification to healthcare facility system 250 once the patient is determined to be in the target area. In some such implementations, healthcare facility system 250 can update the patient's status or location in a patient tracking system. Alternatively, or additionally, services coordination system 230 may update a patient status/location in patient tracker 238.

Optionally, services coordination system 230 may continue to monitor a patient's movement/position in the facility throughout their visit. For example, wearable device 202 may continue to periodically transmit position data even after the patient reaches an initial target area. Periodic or continuous monitoring of patient positions can be useful to a healthcare facility for a number of reasons. Foremost, position monitoring can allow the facility to manage congestion by tracking how many patients are in different areas, how patients are disbursed throughout the facility, which rooms are occupied, etc. Consider, for example, a scenario in which a physician decides that the patient needs an MRI and thus transfers the patient to another area of the facility for receiving the MRI. Services coordination system 230 may be able to track these types of patient movements automatically for keeping patient positions/locations up-to-date. Additionally, patient position tracking can be beneficial in the event of an emergency, as the healthcare facility can quickly determine how many patients are in the building, where patients are located, etc.

Monitoring of a patient's position in the healthcare facility also allows services coordination system 230 to determine when the patient has left the facility/premises, e.g., after their appointment. For example, services coordination system 230 may determine that the patient has left the premises once they are determined to have exited the building (e.g., based on position data from wearable device 202 and/or an IPS) and/or once they are no longer within the facility's geofence. Once it has been determined that the patient has left the facility/premises, services coordination system 230 may optionally provide an indication to healthcare facility system 250. In some implementations, services coordination system 230 and/or healthcare facility system 250 can also update the patient's status and/or remove the patient from the digital queue.

In some implementations, services coordination system 230 further transmits a notification to 202 prompting the patient to submit payment for out-of-pocket expenses (e.g., a copay) related to the visit. In some such implementations, this “out-of-pocket expenses” notification is transmitted after it is determined that the patient has left/exited the healthcare facility. In other implementations, a notification prompting the patient to pay out-of-pocket expenses is transmitted after the patient is checked in for an appointment. In yet other implementations, a notification prompting the patient to pay out-of-pocket expenses is first transmitted after the patient is checked in for an appointment and is subsequently re-transmitted to wearable device 202 after the patient leaves the healthcare facility if the patient does not submit payment responsive to the first request or has an outstanding balance after the appointment.

### Multimodal Patient Management

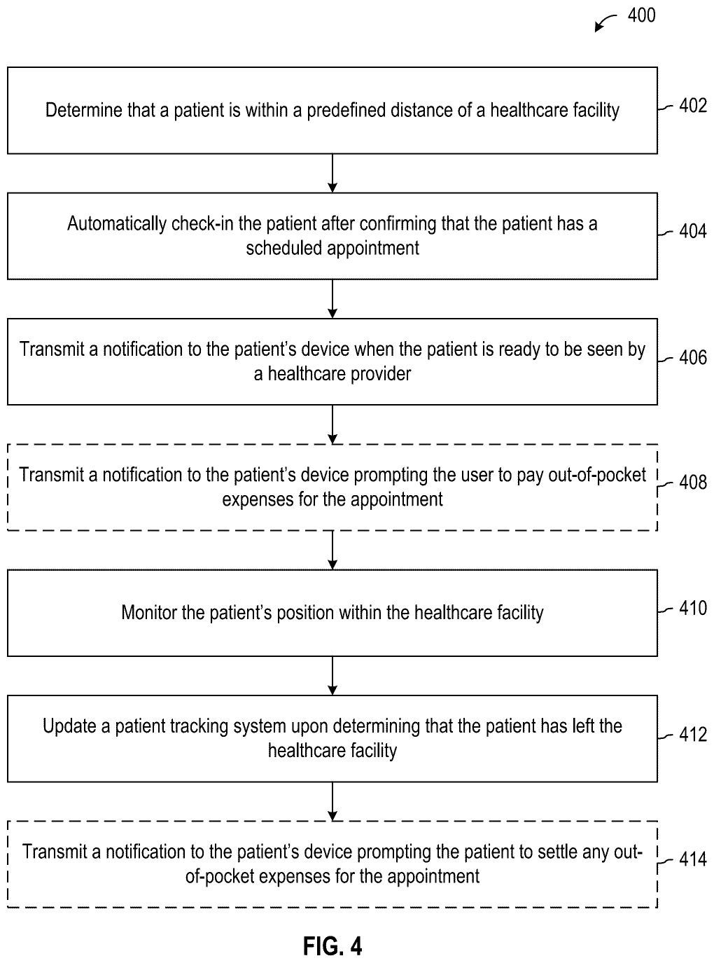

Referring now to FIG. 4, a flow chart of a process 400 for multimodal patient check-in and management is shown, according to some implementations. Unlike existing check-in processes, which are typically manual or require a patient to access a check-in terminal within a healthcare facility, process 400 facilitates automated check-in using wearable devices. In some cases, process 400 is implemented by services coordination system 230, as described above; however, it should be understood that process 400 could be implemented by other suitable devices. It will be appreciated that certain steps of process 400 may be optional and, in some implementations, process 400 may be implemented using less than all of the steps. It will also be appreciated that the order of steps shown in FIG. 4 is not intended to be limiting.

At step 402, it is determined that a patient is within a predefined distance of a healthcare facility. A “predefined distance,” in this case, may be a set distance from the healthcare facility and/or may be defined by a geofence around the healthcare facility. Generally, as discussed above, the determination that the patient is within the threshold distance of the healthcare facility is made based on location data received from a wearable device associated with the patient (e.g., a smartwatch). In some implementations, the location data includes coordinates or the like transmitted from the patient's wearable device. In other implementations, the location data simply includes a notification indicating that the patient is within the threshold distance. In other words, the patient's location may be detected by the wearable device itself. In yet other implementations, the wearable device can be remotely tracked.

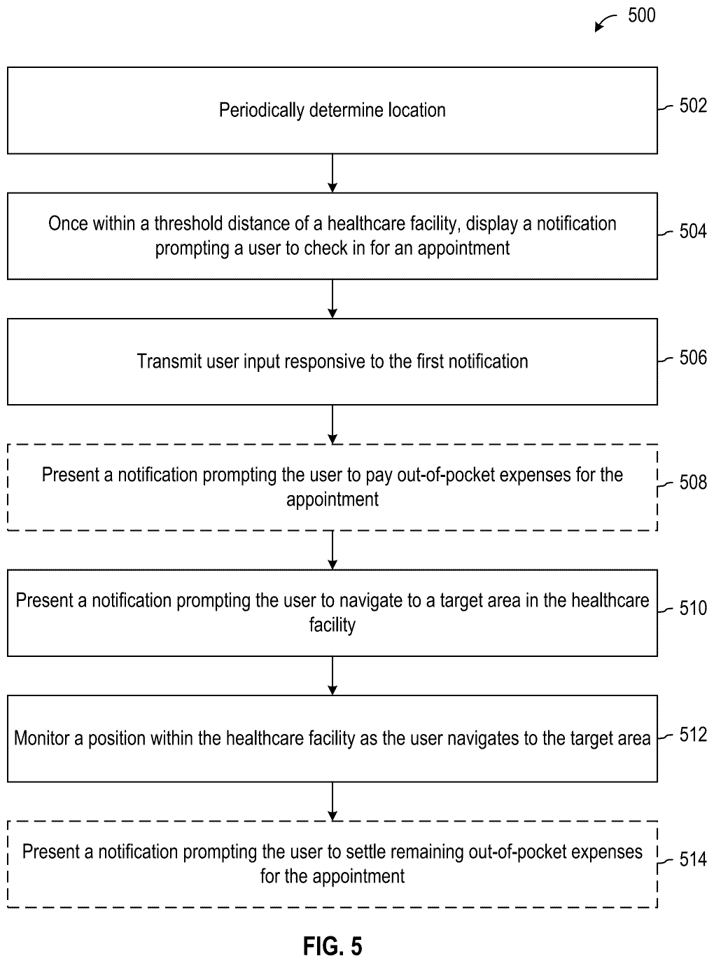

At step 404, the patient is automatically checked in after it is confirmed that the patient has a scheduled appointment. In this regard, prior to step 404, process 400 can include verifying whether the patient has a scheduled appointment at the healthcare facility. For example, identifying information associated with the patient can be used to query a patient scheduling system or healthcare facility scheduling service. In some implementations, the patient is automatically checked in only if a scheduled appointment is verified. However, it should be understood that patients without scheduled appointments may be automatically checked in in certain other implementations. In either case, the patient is typically an existing patient of the healthcare provider and/or a patient that has visited the healthcare facility at least once in the past. In some implementations, as discussed below with respect to FIG. 5, the patient is not automatically checked in until a user input is received from wearable device 202, e.g., approving the automatic check in.

As discussed above, various healthcare providers and/or facilities may have different procedures and/or requirements for patient check-in; thus, the check-in procedure automatically initiated at step 404 may be different among different healthcare providers. For example, patient check-in procedures can include verifying a patient's personal information (e.g., address, phone number, etc.), insurance information, and the like. However, patient check-in generally includes at least indicating a patient status in a patient management system or “digital patient tracker” and/or adding the patient to a digital queue. A “digital patient tracker” or patient management system is typically a system or software program utilized by a healthcare provider to manage patients, including checking-in patients, tracking assigned room numbers, maintaining schedules, booking appointments, etc. In some implementations, automatic check-in includes updating the status of the patient in a digital patient tracking system. In some implementations, automatic check-in includes transmitting a notification indicating that the patient is within the threshold distance of the healthcare facility to a remote system (e.g., healthcare facility system 250) to cause the remote system to automatically perform a check-in procedure and add the patient to a queue.

At step 406, a first notification is transmitted to the patient's device (e.g., wearable device 202) indicating that the patient is ready to be seen by a healthcare provider. The first notification may be transmitted responsive to the patient reaching the “front” of a digital queue and/or the status of the patient being automatically updated once it is determined that the healthcare provider is ready for the appointment. In some implementations, the first notification can be transmitted at a specific time (e.g., at the appointment time). In other implementations, the first notification is transmitted responsive to a user of a digital patient tracker (e.g., a staff member of the healthcare facility) manually updating a status of the patient. In some implementations, receipt of the first notification causes wearable device 202 to display a GUI of the notification.

In addition to indicating that the patient is ready to be seen, the first notification may also indicate a target area for the patient to navigate to within the healthcare facility. For example, the first notification may include a room number for an examination room that the patient has been assigned to. In some implementations, the target area is automatically selected based on the healthcare facility's capacity and/or a disbursement of other patients in the healthcare facility. In some implementations, the target area is manually selected by a user (e.g., of a digital patient tracker of healthcare facility system 250). To this point, it should be appreciated that wearable device 202 may be provided with notifications directing the patient to navigate to other target areas at any time. For example, prior to step 406, the patient may be prompted to navigate to a specific waiting area, e.g., to wait for their appointment.

At step 408, a second notification is optionally transmitted to the patient's device (e.g., wearable device 202) that prompts the patient to pay any out-of-pocket expenses related to the appointment, such as a copay. In some implementations, prior to step 408, it can also be determined whether a patient owes out-of-pocket expenses, e.g., by communicating with healthcare facility system 250 and/or the patient's insurance company. For example, services coordination system 230 may communicate with the patient's insurance provider by providing some details of the visit and obtaining the copay amount. In another example, healthcare facility system 250 may determine whether a copay is required and may provide an indication to services coordination system 230 or wearable device 202 accordingly. While not shown in FIG. 4, it should be appreciated that process 400 can also include facilitating payment of the out-of-pocket expenses if the patient responds to the prompt accordingly. For example, after step 408, an indication of a user input to pay the out-of-pocket expenses may be received from wearable device 202, in which case services coordination system 230 may facilitate an electronic funds transfer and/or otherwise settle the out-of-pocket expenses. In some cases, the patient may choose to “pay later,” in which case the out-of-pocket expenses may be suspended until after the appointment. Should the patient choose to submit payment for the out-of-pocket expenses, services coordination system 230 may utilize stored payment information for the patient (e.g., in a digital wallet) to facilitate payment, e.g., via electronic fund transfer.

At step 410, the patient's position within the healthcare facility is monitored. In other words, the patient is tracked throughout their visit. In some implementations, the patient's position is provided by wearable device 202, e.g., after being determined using an IPS of the healthcare facility. In some such implementations, wearable device 202 regularly (e.g., periodically) transmits position data (e.g., a position in 3D space) to services coordination system 230 for patient tracking. In some implementations, wearable device 202 itself is configured to determine its position within the healthcare facility and can report its location to services coordination system 230. In other implementations, an IPS that is external to wearable device 202 may track the position of wearable device 202. In any case, the patient's location may be reflected in the aforementioned patient tracking system or “digital patient tracker” by updating a patient status and/or record accordingly. For example, the patient's status may be updated to reflect a room or area of the healthcare facility that the patient is occupying.

At step 412, the patient tracking system is updated upon determining that the patient has left the healthcare facility. In this regard, step 412 may coincide with step 410, in which the patient's position/status is periodically updated. In some implementations, the patient is determined to have left the healthcare facility if they are no longer detected using the facility's IPS and/or once the location of wearable device 202 is detected outside of the geofence associated with the facility. Once it is determined that the patient has left the healthcare facility, the patient may be automatically discharged. Similar to check-in procedures, discharge procedures may vary between healthcare providers and/or facilities. But, in any case, the patient's status may be updated to “discharged” and/or to reflect that they are no longer on the premises of the facility. In some implementations, once it is determined that the patient has left the healthcare facility, a notification prompting the patient to schedule a follow-up appointment may be transmitted to wearable device 202 if a follow-up appointment is required. For example, as part of the automated discharge process, services coordination system 230 may determine if the healthcare provider requests a follow-up appointment and may transmit a notification to the patient accordingly. Thus, the patient may be able to schedule or confirm the follow-up appointment directly from their wearable device.

At step 414, a third notification is optionally transmitted to the patient's device that prompts the patient to settle any remaining out-of-pocket expenses related to the appointment. In this regard, step 414 may be implemented if the patient does not submit payment responsive to the notification sent at step 408 and/or does not acknowledge the notification sent at step 408. For example, the patient may choose to “pay later” responsive to the initial prompt for out-of-pocket expenses, in which case the third notification may prompt the patient to settle the out-of-pocket expenses after the appointment. While not shown in FIG. 4, it should be appreciated that process 400 can therefore also include facilitating payment of the out-of-pocket expenses if the patient responds to the prompt accordingly.

Referring now to FIG. 5, a flow chart of a process 500 for multimodal patient check-in and management via a wearable device is shown, according to some implementations. In some cases, process 500 can be implemented by wearable device 202, as described above; however, it should be understood that process 500 could be implemented by other suitable portable computing devices. It will be appreciated that certain steps of process 500 may be optional and, in some implementations, process 500 may be implemented using less than all of the steps. It will also be appreciated that the order of steps shown in FIG. 5 is not intended to be limiting.

At step 502, a location of a user is regularly or periodically determined based location data obtained from a geographical positioning system. As discussed herein, the “user” generally refers to a user of wearable device 202; thus, the location of wearable device 202 is generally indicative of the location of the user since wearable device 202 is configured to be worn by the user when in use. As discussed above, wearable device 202 is generally configured to determine its geographic location using any known geolocating techniques. Commonly, wearable device 202 determines its location using GNSS, GPS, A-GNSS, or A-GPS, e.g., by communicating with navigation satellites and/or cellular towers. Thus, at step 502, wearable device 202 may obtain its location as geographical coordinates; however, the present disclosure is not intended to be limiting in this regard.

At step 504, a first notification prompting the user to check-in for a healthcare appointment is displayed. Generally, as discussed herein, notifications can be presented as GUIs on a display of wearable device 202. In this case, the first notification may be presented as a GUI that asks the user whether they would like to check-in for the appointment (e.g., as shown in FIG. 6A). In some implementations, the first notification is presented responsive to wearable device 202 determining that it is within a threshold distance of a healthcare facility (e.g., defined by a geofence) at which the appointment is scheduled. For example, wearable device 202 may compare its current location (e.g., at each periodic determination per step 502) to the facility's geofence to detect when it is inside the geofenced area. In some such implementations, wearable device 202 can automatically present the first notification once it is determined to be in the geofenced area. In other such implementations, wearable device 202 transmits its determined location to a remote device (e.g., services coordination system 230) which determines whether wearable device 202 is within the threshold distance of the facility. In such implementations, the first notification may be presented responsive to a communication from the remote device.

At step 506, a user input in response to the first notification is transmitted to a remote device. The user input, in this case, may be an indication of user approval to proceed with check-in for the appointment or a user indication that they would prefer to check-in manually. If the user agrees to checking-in automatically, an indication of the user input is transmitted to a remote device to initiate check-in. The remote device may be a computing device or system associated with the healthcare facility (e.g., healthcare facility system 250) or an intermediary patient management device/service (e.g., services coordination system 230). Responsive to the user input, services coordination system 230 or healthcare facility system 250 may initiate an automated check-in procedure, as discussed above, to include updating a status of the user in a digital patient tracker and/or placing the user in a digital queue.

At step 508, a second notification prompting the user to pay out-of-pocket expenses related to the appointment is optionally displayed. Notably, the second notification may be displayed during the automated check-in and/or after the patient is checked in but, in some cases, before the patient's scheduled appointment. In some implementations, the notification simply indicates whether the patient owes for out-of-pocket expenses. For example, services coordination system 230 may determine whether the patient owes for out-of-pocket expenses and may transmit and indication to wearable device 202 accordingly. In other implementations, the notification indicates an amount that the patient owes. In either case, the second notification may be presented responsive to an indication from services coordination system 230 or healthcare facility system 250 that the patient must submit a payment related to the appointment. In some implementations, the user can respond to the second notification by accepting/approving of the payment for the out-of-pocket expenses. In some such implementations, the remote device (e.g., services coordination system 230 or healthcare facility system 250) may facility payment responsive to the user input. Alternatively, in some implementations, the patient may choose to pay the expenses at a later time, in which case the out-of-pocket expenses may be suspended until after the appointment.

At step 510, a third notification prompting the user to navigate to a target area within the healthcare facility is displayed. In this regard, the third notification may indicate the target area to the user. The target area may be, for example, a waiting area, an examination room, or the like. For example, the third notification may prompt the user to “Proceed to Examination Room 102B” for their appointment.

At step 512, a position of the user within the healthcare facility is monitored, e.g., as the user navigates to the target area. As mentioned above, the position of the user within the healthcare facility is typically determined by wearable device 202 using an IPS associated with the facility. For example, the IPS may utilize wireless transceivers (e.g., Wi-Fi routers) or “nodes” positioned throughout the facility for determining the position of wearable device 202, and thereby the user. Alternatively, or additionally, wearable device 202 may utilize GNSS, GPS, A-GNSS, or A-GPS to determine a position within the facility. Thus, in this manner, the user's movement through the healthcare facility can be tracked. In conjunction, the user's position in the healthcare facility may be regularly transmitted to services coordination system 230 or healthcare facility system 250 (e.g., by wearable device 202) such that services coordination system 230 or healthcare facility system 250 can track the user's position in the facility, e.g., to facilitate patient and congestion management.

In some implementations, monitoring of the user's position in the healthcare facility is further used to provide real-time or near real-time directions to the user. In this regard, wearable device 202 may be configured to iteratively calculate a route through the healthcare facility to the target area (e.g., an exam room) based on the user's real-time position. Wearable device 202 may then provide directions to the user in a manner of augmented reality (AR). For example, directions may be displayed via a screen of wearable device 202 in real- or near real-time. In another example, wearable device 202 may transmit directions to another device (e.g., AR glasses or goggles) for display to the user.

At step 514, a fourth notification prompting the user to settle any remaining out-of-pocket expenses related to the appointment is optionally displayed. In this regard, step 514 may be implemented if the patient does not approve of payment responsive to the second notification sent at step 508 and/or if the patient does not acknowledge the notification sent at step 508. For example, the patient may choose to “pay later” responsive to the initial prompt for out-of-pocket expenses, in which case the fourth notification may prompt the patient to settle the out-of-pocket expenses after the appointment (e.g., once it is determined that the patient has left the healthcare facility).

### User Interfaces

Referring now to FIG. 6A-6D, various example user interfaces that can be presented on user device 202 are shown, according to some implementations. FIG. 6A, in particular, shows an example user interface 602 that prompts a user to check-in for an appointment. As mentioned above, user interface 602 may be presented responsive to the user entering a geofence associated with a healthcare facility at which the user has a scheduled appointment. User interface 602—and, more generally, any of the interfaces shown in FIGS. 6A-6D—can include a user-selectable icon (e.g., a digital button) to facilitate user inputs responsive to prompts. In this example, user interface 602 includes an icon that the user can select to check-in for their appointment. FIG. 6B shows another example user interface 604 that can be displayed after the user has been checked in for the appointment. In this example, user interface 604 prompts the user to navigate to “Exam Room 7” in the healthcare facility. In some implementations, user interface 604 is preceded by an interface that indicates to the user that they are ready to be seen by a provider. In other implementations, user interface 604 itself is sufficient to indicate to the user that they are ready to be seen. User interface 604 can also include one or more icons for a user input, including an icon to acknowledge the prompt to navigate to a specific room/area and optionally an icon for signaling to the facility staff that the user is experiencing an emergency.

FIG. 6C shows an example user interface 606 that prompts the user to pay for out-of-pocket expenses related to the appointment. In this example, the user is notified that they owe a $25 copay and is provided with icons for submitting payment immediately or delaying payment (e.g., until after the visit). It should be appreciated that, in some implementations, user interface 606 does not necessarily display an amount owed but may instead simply indicate that the user owes for out-of-pocket expenses. FIG. 6D shows an example user interface 608 for providing live navigation to a user. Specifically, user interface 608 provides directions to the user for navigating through the healthcare facility. In this example, the user is navigating to a specific room in the facility and user interface 606 is displaying directions to the room (e.g., indicating a right turn in the example of FIG. 6D). As discussed above, the directions provided via user interface 608 may be determined using an IPS associated with the healthcare facility.

Referring now to FIG. 7, an example user interface 700 for managing patients in a healthcare facility is shown, according to some implementations. Specifically, user interface 700 is an example of a GUI that can be presented to a staff member at the healthcare facility, or another user, for managing patients manually and/or for viewing/manipulating automated patient management. To this point, in some implementations, user interface 700 can be presented via a computing device at the healthcare facility (e.g., part of healthcare facility system 250). For example, user interface 700 may be presented via a workstation to a staff member at a check-in counter. It should be appreciated that user interface 700 may also be an example of a GUI presented to a user for interacting with services coordination system 230, as described above. For example, user interface 700 may be one of the GUIs that can be accessed via a software program (e.g., executing locally on healthcare facility system 250) associated with services coordination system 230 and/or may be an example webpage or other remotely accessible interface. Thus, the present disclosure is not intended to be limiting in this regard; rather, it will be appreciated that user interface 700 may be accessed or presented in a number of different ways for facilitating user interaction with services coordination system 230 or, more generally, a patient tracking system.

As shown, user interface 700 includes a navigation panel 702 and a patient information panel 704 for tracking patients and/or patient statuses in a healthcare facility. Navigation panel 702 generally allows a user (e.g., healthcare staff) to navigate between various different information in patient information panel 704. Specifically, navigation panel 702 provides a number of different predefined categories or labels relating to specific groups of patients or patient statuses. In this case, the categories/label relate to different patient status for patient tracking. Patient information panel 704 provides various information for the patient associated with a specific category or label. Selecting a specific label causes patient information panel 704 to display information for the associated patients. In the example of FIG. 7, an “Exam Room” category is selected such that patient information panel 704 displays a list of patients that are assigned to an examination room and/or that are currently located in an examination room (e.g., based on positioning data received from wearable devices for each patient). Other categories that are shown include “Appointments,” “Upcoming Appointments,” “Arrived,” “Waiting Room,” and “Discharged.” Thus, it can be appreciated that each category is associated with a patient status relating to a visit to the healthcare facility.

Put in other words, user interface 700 provides a way of managing groups of patients based on their current status and/or location. For example, selecting “Appointments” may display a list of patients that have scheduled appointments or selecting “Arrived” may display a list of patients that are at the healthcare facility (e.g., as determined by location data from respective wearable devices) but not yet checked in. In any case, patient information panel 704 displays information related to any patients in each grouping including a date and/or time that the patient's status was last updated, identifying information for the patient (e.g., name, date of birth, etc.), a reason for the patient's visit, the patient's insurance information, the healthcare provider the patient is scheduled to see, and a current status and/or location of the patient (e.g., which corresponds with the selected label from navigation panel 702). Additionally, patient information panel 704 can include a text entry box 706 in which a user can enter a message to be sent as a notification to the patient. In some implementations, text entry box 706 is configured to accept free text, such that a user can type any message to be transmitted to a patient, e.g., via the patient's wearable device. In some implementations, text entry box 706 is searchable such that the user can select from a plurality of pre-generated messages.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. Various implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A patient management system comprising:
one or more processors; and
memory having instructions stored thereon that, when executed by the one or more processors, cause the system to:
determine that a patient is within a threshold distance of a healthcare facility based on geolocation data for a wearable device associated with the patient, wherein the patient has a scheduled appointment with a healthcare provider at the healthcare facility;
automatically check-in the patient for the scheduled appointment once it is determined that the patient is within the threshold distance of the healthcare facility;
transmit, to the wearable device associated with the patient, a first notification indicating: i) that the patient ready to be seen the healthcare provider for the scheduled appointment, and ii) a target area in the healthcare facility to which the patient is to navigate for the scheduled appointment;
continuously track a position of the patient within the healthcare facility using the wearable device, in part to determine when the patient has exited the healthcare facility; and
update a status of the patient in a digital patient tracker based on the tracked position of the patient within the healthcare facility.

2. The patient management system of claim 1, wherein the geolocation data comprises at least one of assisted global navigation satellite system (A-GNSS) or assisted global positioning system (A-GPS) data obtained by the wearable device.

3. The patient management system of claim 1, wherein to automatically checking-in the patient comprises at least one of to:
update the status of the patient in the digital patient tracker;
add the patient to a digital queue; or
transmit, to a remote computing system associated with the healthcare facility, a third notification indicating that the patient is within the threshold distance of the healthcare facility, wherein the third notification causes the remote computing system to automatically perform a check-in procedure and add the patient to a queue.

4. The patient management system of claim 1, wherein the instructions further cause the system to:
transmit, to the wearable device associated with the patient, a second notification prompting the patient to submit payment of out-of-pocket expenses related to the scheduled appointment.

5. The patient management system of claim 4, wherein the instructions further cause the system to:
receive a user input from the wearable device indicating patient approval of paying the out-of-pocket expenses; and
facilitate the payment of the out-of-pocket expenses using stored payment information associated with the patient.

6. The patient management system of claim 4, wherein second notification is transmitted after the first notification but prior to the patient exiting the healthcare facility.

7. The system of claim 1, wherein the wearable device is configured to display directions for navigating through the healthcare facility to the target area in conjunction with the continuous tracking of the position of the patient within the healthcare facility.

8. The patient management system of claim 1, wherein continuously tracking the position of the patient within the healthcare facility is facilitated by an indoor positioning system (IPS) associated with the healthcare facility.

9. The patient management system of claim 1, wherein the instructions further cause the system to:
periodically transmit updates indicating the position of the patient within the healthcare facility to a remote computing system associated with the healthcare facility to facilitate patient and congestion management.

10. The patient management system of claim 1, wherein the wearable device is a smartwatch.

11. A method of patient management in a healthcare facility, the method comprising:
determining that a patient is within a threshold distance of the healthcare facility based on geolocation data for a wearable device associated with the patient, wherein the patient has a scheduled appointment with a healthcare provider at the healthcare facility;
automatically checking-in the patient for the scheduled appointment once it is determined that the patient is within the threshold distance of the healthcare facility;
transmitting, to the wearable device associated with the patient, a first notification indicating: i) that the patient ready to be seen the healthcare provider for the scheduled appointment, and ii) a target area in the healthcare facility to which the patient is to navigate for the scheduled appointment;
continuously tracking a position of the patient within the healthcare facility using the wearable device, in part to determine when the patient has exited the healthcare facility; and
updating a status of the patient in a digital patient tracker based on the tracked position of the patient within the healthcare facility.

12. The method of claim 11, wherein the geolocation data comprises at least one of assisted global navigation satellite system (A-GNSS) or assisted global positioning system (A-GPS) data obtained by the wearable device.

13. The method of claim 11, wherein to automatically checking-in the patient comprises at least one of:
updating the status of the patient in the digital patient tracker;
adding the patient to a digital queue; or
transmitting, to a remote computing system associated with the healthcare facility, a third notification indicating that the patient is within the threshold distance of the healthcare facility, wherein the third notification causes the remote computing system to automatically perform a check-in procedure and add the patient to a queue.

14. The method of claim 11, further comprising:
transmitting, to the wearable device associated with the patient, a second notification prompting the patient to submit payment of out-of-pocket expenses related to the scheduled appointment.

15. The method of claim 14, further comprising:
receiving a user input from the wearable device indicating patient approval of paying the out-of-pocket expenses; and
facilitating the payment of the out-of-pocket expenses using stored payment information associated with the patient.

16. The method of claim 14, wherein second notification is transmitted after the first notification but prior to the patient exiting the healthcare facility.

17. The method of claim 11, wherein the wearable device is configured to display directions for navigating through the healthcare facility to the target area in conjunction with the continuous tracking of the position of the patient within the healthcare facility.

18. The method of claim 11, wherein continuously tracking the position of the patient within the healthcare facility is facilitated by an indoor positioning system (IPS) associated with the healthcare facility.

19. The method of claim 11, further comprising:
periodically transmitting updates indicating the position of the patient within the healthcare facility to a remote computing system associated with the healthcare facility to facilitate patient and congestion management.

20. A non-transitory computer-readable medium having instructions stored thereon that, when executed by one or more processors, cause a computing device to:
determine that a patient is within a threshold distance of a healthcare facility based on geolocation data for a wearable device associated with the patient, wherein the patient has a scheduled appointment with a healthcare provider at the healthcare facility;
automatically check-in the patient for the scheduled appointment once it is determined that the patient is within the threshold distance of the healthcare facility;
transmit, to the wearable device associated with the patient, a first notification indicating: i) that the patient ready to be seen the healthcare provider for the scheduled appointment, and ii) a target area in the healthcare facility to which the patient is to navigate for the scheduled appointment;
continuously track a position of the patient within the healthcare facility using the wearable device, in part to determine when the patient has exited the healthcare facility; and
update a status of the patient in a digital patient tracker based on the tracked position of the patient within the healthcare facility.
